(19) United States
(12) Patent Application Publication
(10) Pub. No.: US 2006/0069073 A1
(43) Pub. Date: Mar. 30, 2006

(54) Medicaments for inhalation comprising steroids and an anticholinergic

(75) Inventors: Michael P. Pieper, Biberach (DE); Michel Pairet, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/168,619

(22) Filed: Jun. 27, 2005

(30) Foreign Application Priority Data
Jun. 29, 2004 (EP) EP 04015200

(51) Int. Cl. A61K 31/573; A61K 31/4015

(52) U.S. Cl. 514/171; 514/424

(57) ABSTRACT

The present invention relates to novel pharmaceutical compositions based on steroids and salts of an anticholinergic, processes for preparing them and their use in the treatment of respiratory complaints.

This application claims benefit to EP 04015200.1, filed Jun. 26, 2004.

The present invention relates to novel pharmaceutical compositions based on steroids and salts of an anticholinergic of formula 1, 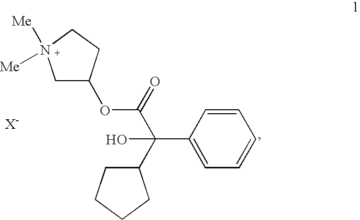
processes for preparing them and their use in the treatment of respiratory complaints.

### DESCRIPTION OF THE INVENTION

The present invention relates to novel pharmaceutical compositions based on steroids and salts of an anticholinergic of formula 1, processes for preparing them and their use in the treatment of respiratory complaints. The compounds of formula 1 are known in the art and also called glycopyrronium salts.

Within the scope of the present invention the anticholinergic agents used are the salts of formula 1 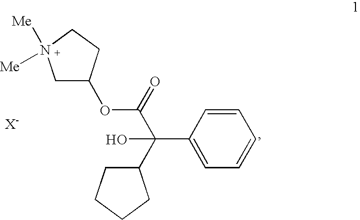
wherein
X⁻ denotes an anion with a single negative charge, preferably an anion selected from the group consisting of fluoride, chloride, bromide, iodide, sulphate, phosphate, methanesulphonate, nitrate, maleate, acetate, citrate, fumarate, tartrate, oxalate, succinate, benzoate and p-toluenesulphonate, optionally in the form of the racemates, the stereoisomers, and the hydrates thereof.

Preferably, the salts of formula 1 are used wherein
X⁻ denotes an anion with a single negative charge selected from among the fluoride, chloride, bromide, 4-toluenesulphonate and methanesulphonate, preferably bromide, optionally in the form of the racemates, the stereoisomers, and the hydrates thereof.

Most preferably, the salts of formula 1 are used wherein
X⁻ denotes an anion with a single negative charge selected from among the chloride, bromide and methanesulphonate, preferably bromide, optionally in the form of the racemates, the stereoisomers, and the hydrates thereof.

Particularly preferred according to the invention is the salt of formula 1 wherein X⁻ denotes bromide.

Of particular interest according to the invention are the stereoisomers of formula (3S,2′R) 1 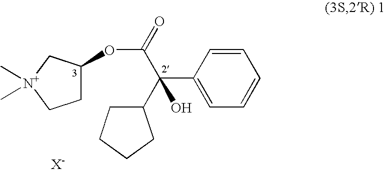
wherein X⁻ may have the meanings an mentioned hereinbefore.

Of particular interest according to the invention are the stereoisomers of formula (3S,2′S) 1 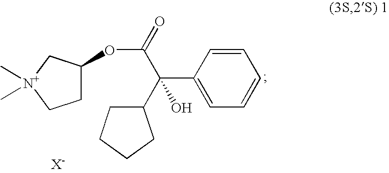
wherein X⁻ may have the meanings as mentioned hereinbefore.

Of particular interest according to the invention are the stereoisomers of formula (3R,2′S)1 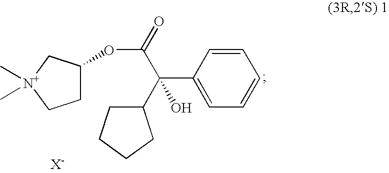
wherein X⁻ may have the meanings as mentioned hereinbefore.

Of particular interest according to the invention are the stereoisomers of formula (3R,2′R) 1 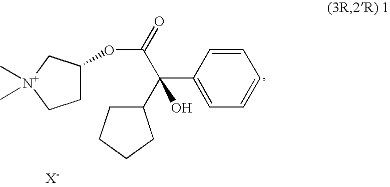
wherein X⁻ may have the meanings an mentioned hereinbefore.

The diasteromers mentioned hereinbefore are obtainable in high optical yield according to methods known in the art (see hereto for instance WO 98/21183). Of particular importance according to the invention are the diasteromers (3R,2′R) 1.

Surprisingly, an unexpectedly beneficial therapeutic effect can be observed in the treatment of inflammatory and/or obstructive diseases of the respiratory tract if the anticholinergic of formula 1 is used with one or more steroids 2.

The beneficial therapeutic effect mentioned above may be observed both when the two active substances are administered simultaneously in a single active substance formulation and when they are administered successively in separate formulations. According to the invention, it is preferable to administer the two active substance ingredients simultaneously in a single formulation.

In the pharmaceutical combinations mentioned above the active substances may be combined in a single preparation or contained in two separate formulations. Pharmaceutical compositions which contain the active substances 1 and 2 in a single preparation are preferred according to the invention.

According to the instant invention preferred steroids 2 which are optionally also referred to as corticosteroids, denote compounds selected from among methyl prednisolone, prednisone, butixocort propionate, RPR-106541, flunisolide, triamcinolone, budesonide, mometasone, ciclesonide, rofleponide, ST-126, dexamethasone, 6α,9α-difluoro-17α-[(2-furanylcarbonyl)oxy]-11β-hydroxy-16α-methyl-3-oxo-androsta-1,4-diene-17β-carbothioic acid (S)-fluoromethyl ester, 6α,9α-difluoro-11β-hydroxy-16α-methyl-3-oxo-17α-propionyloxy-androsta-1,4-diene-17β-carbothioic acid (S)-(2-oxo-tetrahydro-furan-3 S-yl) ester, and etiprednol (BNP-166, hereinafter also referred to as etiprednol-dichloroacetate).

Preferably, the compound 2 is selected from among flunisolide, triamcinolone, budesonide, mometasone, ciclesonide, rofleponide, ST-126, dexamethasone, 6α,9α-difluoro-17α-[(2-furanylcarbonyl)oxy]-11β-hydroxy-16α-methyl-3-oxo-androsta-1,4-diene-17β-carbothioic acid (S)-fluoromethyl ester, 6α,9α-difluoro-11β-hydroxy-16α-methyl-3-oxo-17α-propionyloxy-androsta-1,4-diene-17β-carbothioic acid (S)-(2-oxo-tetrahydro-furan-3S-yl) ester, and etiprednol. More preferably, the compound 2 is selected from among budesonide, mometasone, ciclesonide, 6α,9α-difluoro-17α-[(2-furanylcarbonyl)oxy]-11β-hydroxy-16α-methyl-3-oxo-androsta-1,4-diene-17β-carbothioic acid (S)-fluoromethyl ester, and etiprednol-dichloroacetat, more preferably the compound 2 is selected from among budesonide, mometasone, ciclesonide, and etiprednol.

Any reference to steroids 2 within the scope of the present invention includes a reference to the salts or derivatives which may be formed from the steroids. Examples of possible salts or derivatives include: sodium salts, sulphobenzoates, phosphates, isonicotinates, acetates, dichloroacetates, propionates, dihydrogen phosphates, palmitates, pivalates or furoates. In some cases the compounds of formula 2 may also occur in the form of their hydrates. Any reference to steroids 2 within the scope of the present invention also includes a reference to the compounds 2 in the form of their diastereomers, mixtures of diastereomers or in the form of the racemates.

In one aspect the present invention relates to the abovementioned pharmaceutical compositions which contain, in addition to therapeutically effective quantities of 1 and 2, a pharmaceutically acceptable carrier. In another aspect the present invention relates to the abovementioned pharmaceutical compositions which do not contain any pharmaceutically acceptable carrier in addition to therapeutically effective quantities of 1 and 2.

In one aspect the present invention relates to the above-mentioned medicament combinations which contain in addition to therapeutically effective amounts of 1 and 2 a pharmaceutically acceptable carrier. In one aspect the present invention relates to the above-mentioned pharmaceutical compositions which do not contain contain a pharmaceutically acceptable carrier in addition to therapeutically effective amounts of 1 and 2 .

The present invention also relates to the use of therapeutically effective amounts of the active substances 1 for preparing a pharmaceutical composition also containing one or more, preferably one active substance 2 for the treatment of inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) as well as for the treatment of skin irritations and inflammation.

In a preferred aspect the present invention relates to the use of therapeutically effective amounts of the active substance 1 for preparing a pharmaceutical composition also containing one or more, preferably one, active substance 2 for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament combinations according to the invention are used as specified above for preparing a pharmaceutical composition for the treatment of obstructive pulmonary diseases selected from among bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma and COPD.

It is also preferable to use the medicament combinations according to the invention for preparing a pharmaceutical composition for the treatment of pulmonary emphysema which has its origins in COPD (chronic obstructive pulmonary disease) or α1-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament combinations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as for example bronchitis caused by bacterial or viral infection, allergic bronchitis and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary oedema, for example toxic pulmonary oedema after aspiration or inhalation of toxic substances and foreign substances.

It is particularly preferable to use the compounds detailed above for preparing a pharmaceutical composition for the treatment of asthma or COPD. Also of particular importance is the above-mentioned use of medicament combinations according to the invention for preparing a pharmaceutical composition for once-a-day treatment of inflammatory and obstructive respiratory complaints, particularly for the once-a-day treatment of asthma or COPD.

The present invention also relates to the use of therapeutically effective amounts of an active substance of formula 1 in combination with therapeutically effective amounts of active substance 2 for preparing a pharmaceutical composition for the treatment of one of the above-mentioned diseases.

The present invention also relates to a process for treating one of the above-mentioned diseases, which is characterised in that therapeutically effective amounts of active substance of formula 1 are administered in combination with therapeutically effective amounts of active substance 2.

Within the scope of the present invention the compounds 1 and 2 may be administered simultaneously or successively, while it is preferable according to the invention to administer compounds 1 and 2 simultaneously.

The present invention further relates to the use of therapeutically effect amounts of salts 1 and steroids 2 for treating inflammatory or obstructive respiratory complaints, particularly asthma or COPD.

The proportions in which the active substances 1 and 2 may be used in the active substance combinations according to the invention are variable. Active substances 1 and 2 may possibly be present in the form of their solvates or hydrates. Depending on the choice of the compounds 1 and 2 the weight ratios which may be used within the scope of the present invention vary on the basis of the different molecular weights of the various compounds and their different potencies. As a rule, the pharmaceutical combinations according to the invention may contain the cation 1 and a steroid 2 in ratios by weight ranging from 1:250 to 250:1, preferably from 1:150 to 150:1. In the particularly preferred pharmaceutical combinations which contain in addition to 1 a compound selected for instance from among the group consisting of budesonide, mometasone, and ciclesonide as the steroid 2, the weight ratios of 1 to 2 are most preferably in a range from about 1:40 to 40: 1, more preferably from 1:30 to 30:1.

For example, without restricting the scope of the invention thereto, preferred combinations of 1 and 2 according to the invention may contain 1 and one of the abovementioned preferred steroids 2 in the following weight ratios: 1:65, 1:64, 1:63, 1:62, 1:61, 1:60, 1:59, 1:58, 1:57, 1:56, 1:55, 1:54, 1:53, 1:52, 1:51, 1:50; 1:49; 1:48; 1:47; 1:46; 1:45; 1:44; 1:43; 1:42; 1:41; 1:40; 1:39; 1:38; 1:37; 1:36; 1:35; 1:34; 1:33; 1:32; 1:31; 1:30; 1:29; 1:28; 1:27; 1:26; 1:25; 1:24; 1:23; 1:22; 1:21; 1:20; 1:19; 1:18; 1:17; 1:16; 1:15; 1:14; 1:13; 1:12; 1:11; 1:10; 1:9; 1:8; 1:7; 1:6; 1:5; 1:4; 1:3; 1:2; 1:1; 2:1; 3:1; 4:1; 5:1; 6:1; 7:1; 8:1; 9:1; 10:1; 11:1; 12:1; 13:1; 14:1; 15:1; 16:1; 17:1; 18:1; 19:1; 20:1; 21:1; 22:1; 23:1; 24:1; 25:1; 26:1; 27:1; 28:1; 29:1; 30:1.

The pharmaceutical compositions according to the invention containing the combinations of 1 and 2 are normally administered so that 1 and 2 are present together in doses of 0.1 to 10000 μg, preferably from 1 to 5000 μg, more preferably from 10 to 3000 μg, better still from 100 to 2000 μg per single dose. For example, combinations of 1 and 2 according to the invention contain a quantity of 1 and steroid 2 (as for instance budesonide, mometasone, ciclesonide) such that the total dosage per single dose is about 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg 935 μg, 940 μg, 945 μg, 950 μg, 955 μg, 960 μg, 965 μg, 970 μg, 975 μg, 980 μg, 985 μg, 990 μg, 995 μg, 1000 μg, 1005 μg, 1010 μg, 1015 μg, 1020kg, 1025 μg, 1030 μg, 1035 μg, 1040 μg, 1045 μg, 1050 μg, 1055 μg, 1060 μg, 1065 μg, 1070 μg, 1075 μg, 1080 μg, 1085 μg, 1090 μg, 1095 μg, 1100 μg, 1105 μg, 1110 μg, 1115 μg, 1120 μg, 1125 μg, 1130 μg, 1135 μg, 1140 μg, 1145 μg, 1150 μg, 1155 μg, 1160 μg, 1165 μg, 1170 μg, 1175 μg, 1180 μg, 1185 μg, 1190 μg, 1195 μg, 1200 μg, 1205 μg, 1210 μg, 1215 μg, 1220 μg, 1225 μg, 1230 μg, 1235 μg, 1240 μg, 1245 μg, 1250 μg, 1255 μg, 1260 μg, 1265 μg, 1270 μg, 1275 μg, 1280 μg, 1285 μg, 1290 μg, 1295 μg, 1300 μg, 1305 μg, 1310 μg, 1315 μg, 1320 μg, 1325 μg, 1330 μg, 1335 μg, 1340 μg, 1345 μg, 1350 μg, 1355 μg, 1360 μg, 1365 μg, 1370 μg, 1375 μg, 1380 μg, 1385 μg, 1390 μg, 1395 μg, 1400 μg, 1405 μg, 1410 μg, 1415 μg, 1420 μg, 1425 μg, 1430 μg, 1435 μg, 1440 μg, 1445 μg, 1450 μg, 1455 μg, 1460 μg, 1465 μg, 1470 μg, 1475 μg, 1480 μg, 1485 μg, 1490 μg, 1495 μg, 1500 μg, 1505 μg, 1510 μg, 1515 μg, 1520 μg, 1525 μg, 1530 μg, 1535 μg, 1540 μg, 1545 μg, 1550 μg, 1555 μg, 1560 μg, 1565 μg, 1570 μg, 1575 μg, 1580 μg, 1585 μg, 1590 μg, 1595 μg, 1600 μg, 1605 μg, 1610 μg, 1615 μg, 1620 μg, 1625 μg, 1630 μg, 1635 μg, 1640 μg, 1645 μg, 1650 μg, 1655 μg, 1660 μg, 1665 μg, 1670 μg, 1675 μg, 1680 μg, 1685 μg, 1690 μg, 1695 μg, 1700 μg, 1705 μg, 1710 μg, 1715 μg, 1720 μg, 1725 μg, 1730 μg, 1735 μg, 1740 μg, 1745 μg, 1750 μg, 1755 μg, 1760 μg, 1765 μg, 1770 μg, 1775 μg, 1780 μg, 1785 μg, 1790 μg, 1795 μg, 1800 μg, 1805 μg, 1810 μg, 1815 μg, 1820 μg, 1825 μg, 1830 μg, 1835 μg, 1840 μg, 1845 μg, 1850 μg, 1855 μg, 1860 μg, 1865 μg, 1870 μg, 1875 μg, 1880 μg, 1885 μg, 1890 μg, 1895 μg, 1900 μg, 1905 μg, 1910 μg, 1915 μg, 1920 μg, 1925 μg, 1930 μg, 1935 μg, 1940 μg, 1945 μg, 1950 μg, 1955 μg, 1960 μg, 1965 μg, 1970 μg, 1975 μg, 1980 μg, 1985 μg, 1990μg, 1995 μg, 2000 μg or similar. It is clear to anyone skilled in the art that the suggested dosages per single dose specified above are not to be regarded as being limited to the numerical values actually stated. Fluctuations of about±2.5 μg, particularly in the decimal range, are also included, as will be apparent to the skilled man. In these dosage ranges, the active substances 1 and 2 may be present in the weight ratios given above.

For example, without restricting the scope of the invention thereto, the combinations of 1 and 2 according to the invention may contain a quantity of cation 1 (if for instance the bromide is used as the preferred combination partner) and steroid 2 (as for instance budesonide, mometasone, ciclesonide, etiprednol) such that, for each single dose 40 μg of 1 and 25 μg of 2, 40 μg of 1 and 50 μg of 2, 40 μg of 1 and 100 μg of 2, 40 μg of 1 and 200 μg of 2, 40 μg of 1 and 300 μg of 2, 40 μg of 1 and 400 μg of 2, 40 μg of 1 and 500 μg of 2, 40 μg of 1 and 600 μg of 2, 40 μg of 1 and 700 μg of 2, 40 μg of 1 and 800 μg of 2, 40 μg of 1 and 900 μg of 2, 40 μg of 1 and 1000 μg of 2, 60 μg of 1 and 25 μg of 2, 60 μg of 1 and 50 μg of 2, 60 μg of 1 and 100 μg of 2, 60 μg of 1 and 200 μg of 2, 60 μg of 1 and 300 μg of 2, 60 μg of 1 and 400 μg of 2, 60 μg of 1 and 500 μg of 2, 60 μg of 1 and 600 μg of 2, 60 μg of 1 and 700 μg of 2, 60 μg of 1 and 800 μg of 2, 60 μg of 1 and 900 μg of 2, 60 μg of 1 and 1000 μg of 2, 80 μg of 1 and 25 μg of 2, 80 μg of 1 and 50 μg of 2, 80 μg of 1 and 100 μg of 2, 80 μg of 1 and 200 μg of 2, 80 μg of 1 and 300 μg of 2, 80 μg of 1 and 400 μg of 2, 80 μg of 1 and 500 μg of 2, 80 μg of 1 and 600 μg of 2, 80 μg of 1 and 700 μg of 2, 80 μg of 1 and 800 μg of 2, 80 μg of 1 and 900 μg of 2, 80 μg of 1 and 1000 μg of 2, 100 μg of 1 and 25 μg of 2, 100 μg of 1 and 50 μg of 2, 100 μg of 1 and 100 μg of 2, 100 μg of 1 and 200 μg of 2, 100 μg of 1 and 300 μg of 2, 100 μg of 1 and 400 μg of 2, 100 μg of 1 and 500 μg of 2, 100 μg of 1 and 600 μg of 2, 100 μg of 1 and 700 μg of 2, 100 μg of 1 and 800 μg of 2, 100 μg of 1 and 900 μg of 2, 100 μg of 1 and 1000 μg of 2, 150 μg of 1 and 25 μg of 2, 150 μg of 1 and 50 μg of 2, 150 μg of 1 and 100 μg of 2, 150 μg of 1 and 200 μg of 2, 150 μg of 1 and 300 μg of 2, 150 μg of 1 and 400 μg of 2, 150 μg of 1 and 500 μg of 2, 150 μg of 1 and 600 μg of 2, 150 μg of 1 and 700 μg of 2, 150 μg of 1 and 800 μg of 2, 150 μg of 1 and 900 μg of 2, 150 μg of 1 and 1000 μg of 2, 200 μg of 1 and 25 μg of 2, 200 μg of 1 and 50 μg of 2, 200 μg of 1 and 100 μg of 2, 200 μg of 1 and 200 μg of 2, 200 μg of 1 and 300 μg of 2, 200 μg of 1 and 400 μg of 2, 200 μg of 1 and 500 μg of 2, 200 μg of 1 and 600 μg of 2, 200 μg of 1 and 700 μg of 2, 200 μg of 1 and 800 μg of 2, 200 μg of 1 and 900 μg of 2, 200 μg of 1 and 1000 μg of 2, 250 μg of 1 and 25 μg of 2, 250 μg of 1 and 50 μg of 2, 250 μg of 1 and 100 μg of 2, 250 μg of 1 and 200 μg of 2, 250 μg of 1 and 300 μg of 2, 250 μg of 1 and 400 μg of 2, 250 μg of 1 and 500 μg of 2, 250 μg of 1 and 600 μg of 2, 250 μg of 1 and 700 μg of 2, 250 μg of 1 and 800 μg of 2, 250 μg of 1 and 900 μg of 2, 250 μg of 1 and 1000 μg of 2, 400 μg of 1 and 25 μg of 2, 400 μg of 1 and 50 μg of 2, 400 μg of 1 and 100 μg of 2, 400 μg of 1 and 200 μg of 2, 400 μg of 1 and 300 μg of 2, 400 μg of 1 and 400 μg of 2, 400 μg of 1 and 500 μg of 2, 400 μg of 1 and 600 μg of 2, 400 μg of 1 and 700 μg of 2, 400 μg of 1 and 800 μg of 2, 400 μg of 1 and 900 μg of 2 or 400 μg of 1 and 1000 μg of 2, 500 μg of 1 and 25 μg of 2, 500 μg of 1 and 50 μg of 2, 500 μg of 1 and 100 μg of 2, 500 μg of 1 and 200 μg of 2, 500 μg of 1 and 300 μg of 2, 500 μg of 1 and 400 μg of 2, 500 μg of 1 and 500 μg of 2, 500 μg of 1 and 600 μg of 2, 500 μg of 1 and 700 μg of 2, 500 μg of 1 and 800 μg of 2, 500 μg of 1 and 900 μg of 2 or 500 μg of 1 and 1000 μg of 2, 600 μg of 1 and 25 μg of 2, 600 μg of 1 and 50 μg of 2, 600 μg of 1 and 100 μg of 2, 600 μg of 1 and 200 μg of 2, 600μg of 1 and 300 μg of 2, 600 μg of 1 and 400 μg of 2, 600 μg of 1 and 500 μg of 2, 600 μg of 1 and 600 μg of 2, 600 μg of 1 and 700 μg of 2, 600 μg of 1 and 800 μg of 2, 600 μg of 1 and 900 μg of 2 or 600 μg of 1 and 1000 μg of 2, 700 μg of 1 and 25 μg of 2, 700 μg of 1 and 50 μg of 2, 700 μg of 1 and 100 μg of 2, 700 μg of 1 and 200 μg of 2, 700 μg of 1 and 300 μg of 2, 700 μg of 1 and 400 μg of 2, 700 μg of 1 and 500 μg of 2, 700 μg of 1 and 600 μg of 2, 700 μg of 1 and 700 μg of 2, 700 μg of 1 and 800 μg of 2, 700μg of 1 and 900 μg of 2, 700 μg of 1 and 1000 μg of 2, 800 μg of 1 and 25 μg of 2, 800 μg of 1 and 50 μg of 2, 800 μg of 1 and 100 μg of 2, 800 μg of 1 and 200 μg of 2, 800 μg of 1 and 300 μg of 2, 800 μg of 1 and 400 μg of 2, 800 μg of 1 and 500 μg of 2, 800 μg of 1 and 600 μg of 2, 800 μg of 1 and 700 μg of 2, 800 μg of 1 and 800 μg of 2, 800 μg of 1 and 900 μg of 2, 800 μg of 1 and 1000 μg of 2, 900 μg of 1 and 25 μg of 2, 900 μg of 1 and 50 μg of 2, 900 μg of 1 and 100 μg of 2, 900 μg of 1 and 200 μg of 2, 900 μg of 1 and 300 μg of 2, 900 μg of 1 and 400 μg of 2, 900 μg of 1 and 500 μg of 2, 900 μg of 1 and 600 μg of 2, 900 μg of 1 and 700 μg of 2, 900 μg of 1 and 800 μg of 2, 900 μg of 1 and 900 μg of 2, 900 μg of 1 and 1000 μg of 2, 1000 μg of 1 and 25 μg of 2, 1000 μg of 1 and 50 μg of 2, 1000 μg of 1 and 100 μg of 2, 1000 μg of 1 and 200 μg of 2, 1000 μg of 1 and 300 μg of 2, 1000 μg of 1 and 400 μg of 2, 1000 μg of 1 and 500 μg of 2, 1000 μg of 1 and 600 μg of 2, 1000 μg of 1 and 700 μg of 2, 1000 μg of 1 and 800 μg of 2, 1000 μg of 1 and 900 μg of 2 or 1000 μg of 1 and 1000 μg of 2 are present.

The active substance combinations of 1 and 2 according to the invention are preferably administered by inhalation. For this purpose, ingredients 1 and 2 have to be made available in forms suitable for inhalation. Inhalable preparations according to the invention include inhalable powders, propellant-containing metered dose aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term carrier may optionally be used instead of the term excipient. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

A) Inhalable Powder Containing the Combinations of Active Substances 1 and 2 According to the Invention:

The inhalable powders according to the invention may contain 1 and 2 either on their own or in admixture with suitable physiologically acceptable excipients.

If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextran), polyalcohols (e.g. sorbitol, mannitol, xylitol), cyclodextrines (e.g. α-cyclodextrine, β-cyclodextrine, X-cyclodextrine, methyl-β-cyclodextrine, hydroxypropyl-β-cyclodextrine), salts (e.g. sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 6 μm, is added to the excipient mixture. Processes for producing the inhalable powders according to the invention by grinding and micronising and by finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain one or more physiologically acceptable excipients in addition to 1 and 2 may be administered, for example, by means of inhalers which deliver a single dose from a supply using a measuring chamber as described in U.S. Pat. No. 4,570,630A, or by other means as described in DE 36 25 685 A. The inhalable powders according to the invention which contain 1 and 2 optionally in conjunction with a physiologically acceptable excipient may be administered, for example, using the inhaler known by the name Turbuhaler® or using inhalers as disclosed for example in EP 237507 A. Preferably, the inhalable powders according to the invention which contain physiologically acceptable excipient in addition to 1 and 2 are packed into capsules (to produce so-called inhalettes) which are used in inhalers as described, for example, in WO 94/28958.

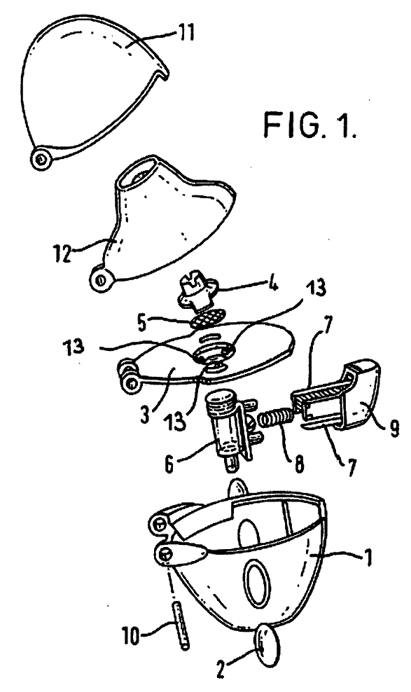

A particularly preferred inhaler for using the pharmaceutical combination according to the invention in inhalettes is shown in FIG. 1.

This inhaler (Handyhaler) for inhaling powdered pharmaceutical compositions from capsules is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, as well as airholes 13 for adjusting the flow resistance.

If the inhalable powders according to the invention are packed into capsules (inhalers) for the preferred use described above, the quantities packed into each capsule should be 1 to 30 mg per capsule. These capsules contain, according to the invention, either together or separately, the doses of 1 or 2 mentioned hereinbefore for each single dose.

B) Propellant Gas-Driven Inhalation Aerosols Containing the Combinations of Active Substances 1 and 2:

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane) and TG227 (1,1,1,2,3,3,3-heptafluoropropane) and mixtures thereof, of which the propellant gases TG134a, TG227 and mixtures thereof are preferred.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention. The present invention also relates to cartridges fitted with a suitable valve which can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Active Substances 1 and 2 According to the Invention:

Propellant-free inhalable solutions and suspensions according to the invention contain, for example, aqueous or alcoholic, preferably ethanolic solvents, optionally ethanolic solvents mixed with aqueous solvents. If aqueous/ethanolic solvent mixtures are used the relative proportion of ethanol compared with water is not limited but preferably the maximum is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of editic acid (EDTA) or one of the known salts thereof, sodium editate, as stabiliser or complexing agent is unnecessary in the present formulation. Other embodiments may contain this compound or these compounds. In a preferred embodiment the content based on sodium editate is less than 100 mg/100 ml, preferably less than 50 mg/100 ml, more preferably less than 20 mg/100 ml. Generally, inhalable solutions in which the content of sodium editate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents. The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium editate. In another preferred embodiment, no sodium editate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 20 and 30 μL of active substance solution can be nebulised in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

This nebuliser (Respimat®) can advantageously be used to produce the inhalable aerosols according to the invention containing the combination of active substances 1 and 2.

Because of its cylindrical shape and handy size of less than 9 to 15 cm long and 2 to 4 cm wide, this device can be carried at all times by the patient. The nebuliser sprays a defined volume of pharmaceutical formulation using high pressures through small nozzles so as to produce inhalable aerosols.

The preferred atomiser essentially consists of an upper housing part, a pump housing, a nozzle, a locking mechanism, a spring housing, a spring and a storage container, characterised by
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing,
a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 Mpa (about 50 to 600 bar), preferably 10 to 60 Mpa (about 100 to 600 bar) on the fluid, the measured amount of active substance solution, at its high pressure end at the moment when the spring is actuated. Volumes of 10 to 50 microlitres are preferred, while volumes of 10 to 20 microlitres are particularly preferred and a volume of 15 microlitres per spray is most particularly preferred.

The valve body is preferably mounted at the end of the hollow plunger facing the valve body.

The nozzle in the nozzle body is preferably microstructured, i.e. produced by microtechnology. Microstructured nozzle bodies are disclosed for example in WO 94/07607; reference is hereby made to the contents of this specification, particularly FIG. 1 therein and the associated description.

The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20 to 160° to one another, preferably 60 to 150°, most preferably 80 to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 to 300 bar, and is atomised into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomiser axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomiser; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomiser is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g. 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomised may be pushed into the atomiser one after another and used in succession. The storage container contains the aqueous aerosol preparation according to the invention. The atomising process is initiated by pressing gently on the actuating button. As a result, the locking mechanism opens up the path for the power takeoff member. The biased spring pushes the plunger into the cylinder of the pump housing. The fluid leaves the nozzle of the atomiser in atomised form.

Further details of construction are disclosed in PCT Applications WO 97/12683 and WO 97/20590, to which reference is hereby made.

The components of the atomiser (nebuliser) are made of a material which is suitable for its purpose. The housing of the atomiser and, if its operation permits, other parts as well, are preferably made of plastics, e.g. by injection moulding. For medicinal purposes, physiologically safe materials are used.

FIGS. 6a/b of WO 97/12687, show the nebuliser (Respimat®) which can advantageously be used for inhaling the aqueous aerosol preparations according to the invention.

FIG. 6a of WO 97/12687 shows a longitudinal section through the atomiser with the spring biased while FIG. 6b of WO 97/12687 shows a longitudinal section through the atomiser with the spring relaxed.

The upper housing part (51) contains the pump housing (52) on the end of which is mounted the holder (53) for the atomiser nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomised. The storage container is sealed off by the stopper (73) through which the hollow plunger projects into the storage container and is immersed at its end in the fluid (supply of active substance solution). The spindle (74) for the mechanical counter is mounted in the covering of the spring housing. At the end of the spindle facing the upper housing part is the drive pinion (75). The slider (76) sits on the spindle.

The nebuliser described above is suitable for nebulising the aerosol preparations according to the invention to produce an aerosol suitable for inhalation.

If the formulation according to the invention is nebulised using the method described above (Respimat®) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defined mass on each actuation.

However, the formulation according to the invention may also be nebulised by means of inhalers other than those described above, e.g. jet stream inhalers or other stationary nebulisers.

Accordingly, in a further aspect, the invention relates to pharmaceutical formulations in the form of propellant-free inhalable solutions or suspensions as described above combined with a device suitable for administering these formulations, preferably in conjunction with the Respimat®. Preferably, the invention relates to propellant-free inhalable solutions or suspensions characterised by the combination of active substances 1 and 2 according to the invention in conjunction with the device known by the name Respimat®. In addition, the present invention relates to the above-mentioned devices for inhalation, preferably the Respimat®, characterised in that they contain the propellant-free inhalable solutions or suspensions according to the invention as described hereinbefore.

According to the invention, inhalable solutions which contain the active substances 1 and 2 in a single preparation are preferred. The term “single preparation” also includes preparations which contain the two ingredients 1 and 2 in two-chamber cartridges, as disclosed for example in WO 00/23037. Reference is hereby made to this publication in its entirety.

The propellant-free inhalable solutions or suspensions according to the invention may take the form of concentrates or sterile inhalable solutions or suspensions ready for use, as well as the above-mentioned solutions and suspensions designed for use in a Respimat®. Formulations ready for use may be produced from the concentrates, for example, by the addition of isotonic saline solutions. Sterile formulations ready for use may be administered using energy-operated free-standing or portable nebulisers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterised in that the device is an energy-operated free-standing or portable nebuliser which produces inhalable aerosols by means of ultrasound or compressed air by the Venturi principle or other methods.

The Examples which follow serve to illustrate the present invention in more detail without restricting the scope of the invention to the following embodiments by way of example.

### EXAMPLES OF FORMULATIONS

The following examples of formulations, which may be obtained analogously to methods known in the art, serve to illustrate the present invention more fully without restricting it to the contents of these examples. In the following exmples the stereoisomerically pure forms are used as the active ingredient.

Inhalable Powders Racemate:

| | |
|---|---|
| Ingredients | μg per capsule |

1)

| | |
|---|---|
| Racemate I (bromide) | 60 |
| budesonide | 200 |
| lactose | 12240 |
| Total | 12500 |

2)

| | |
|---|---|
| Racemate I (bromide) | 60 |
| Eiprednol-dichloroacetat | 125 |
| lactose | 4815 |
| Total | 5000 |

3)

| | |
|---|---|
| Racemate I (bromide) | 60 |
| Mometasone-furoate X H₂O | 250 |
| lactose | 12240 |
| Total | 12500 |

4)

| | |
|---|---|
| Racemate I (bromide) | 60 |
| Ciclesonide | 250 |
| lactose | 12240 |
| Total | 12500 |

5)

| | |
|---|---|
| Racemate I (bromide) | 100 |
| budesonide | 125 |
| lactose | 4775 |
| Total | 5000 |

6)

| | |
|---|---|
| Racemate I (bromide) | 100 |
| Etiprednol-dichloroacetate | 200 |
| lactose | 4700 |
| Total | 5000 |

7)

| | |
|---|---|
| Racemate I (bromide) | 100 |
| Mometasone-furoate X H₂O | 250 |
| lactose | 4650 |
| Total | 5000 |

8)

| | |
|---|---|
| Racemate I (bromide) | 100 |
| Ciclesonide | 250 |
| lactose | 4650 |
| Total | 5000 |

B) Propellant-Containing Aerosols for Inhalation:

| | |
|---|---|
| Ingredients | % by weight |

1)

| | |
|---|---|
| Racemate I (bromide) | 0.15 |
| budesonide | 0.4 |
| soya lecithin | 0.2 |
| TG 134a:TG227 = 2:3 | ad 100 |

2)

| | |
|---|---|
| Racemate I (bromide) | 0.2 |
| Etiprednol-dichloroacetate | 0.3 |
| Isopropyl myristate | 0.1 |
| TG 227 | ad 100 |

3)

| | |
|---|---|
| Racemate I (bromide) | 0.15 |
| Mometasone-furoate X H₂O | 0.6 |
| Isopropyl myristate | 0.1 |
| TG 227 | ad 100 |

4)

| | |
|---|---|
| Racemate I (bromide) | 0.2 |
| Ciclesonide | 0.4 |
| Isopropyl myristate | 0.1 |
| TG 134a:TG227 = 2:3 | ad 100 |

Inhalable Powders (3S,2′R):

| | |
|---|---|
| Ingredients | μg per capsule |

1)

| | |
|---|---|
| (3S,2′R) I (bromide) | 60 |
| budesonide | 200 |
| lactose | 12240 |
| Total | 12500 |

2)

| | |
|---|---|
| (3S,2′R) I (bromide) | 60 |
| Etiprednol-dichloroacetate | 125 |
| lactose | 4815 |
| Total | 5000 |

3)

| | |
|---|---|
| (3S,2′R) I (bromide) | 60 |
| Mometasone-furoate X H₂O | 250 |
| lactose | 12240 |
| Total | 12500 |

4)

| | |
|---|---|
| (3S,2′R) I (bromide) | 60 |
| Ciclesonide | 250 |
| lactose | 12240 |
| Total | 12500 |

5)

| | |
|---|---|
| (3S,2′R) I (bromide) | 100 |
| budesonide | 125 |
| lactose | 4775 |
| Total | 5000 |

6)

| | |
|---|---|
| (3S,2′R) I (bromide) | 100 |
| Etiprednol-dichloroacetate | 200 |
| lactose | 4700 |
| Total | 5000 |

7)

| | |
|---|---|
| (3S,2′R) I (bromide) | 100 |
| Mometasone-furoate X H₂O | 250 |
| lactose | 4650 |
| Total | 5000 |

8)

| | |
|---|---|
| (3S,2′R) I (bromide) | 100 |
| Ciclesonide | 250 |
| lactose | 4650 |
| Total | 5000 |

B) Propellant-Containing Aerosols for Inhalation:

| | |
|---|---|
| Ingredients | % by weight |

1)

| | |
|---|---|
| (3S,2′R) I (bromide) | 0.15 |
| budesonide | 0.4 |
| soya lecithin | 0.2 |
| TG 134a:TG227 = 2:3 | ad 100 |

2)

| | |
|---|---|
| (3S,2′R) I (bromide) | 0.2 |
| Etiprednol-dichloroacetate | 0.3 |
| Isopropyl myristate | 0.1 |
| TG 227 | ad 100 |

3)

| | |
|---|---|
| (3S,2′R) I (bromide) | 0.15 |
| Mometasone-furoate X H₂O | 0.6 |
| Isopropyl myristate | 0.1 |
| TG 227 | ad 100 |

4)

| | |
|---|---|
| (3S,2′R) I (bromide) | 0.2 |
| Ciclesonide | 0.4 |
| Isopropyl myristate | 0.1 |
| TG 134a:TG227 = 2:3 | ad 100 |

Inhalable Powders (3S,2′S):

| | |
|---|---|
| Ingredients | μg per capsule |

1)

| | |
|---|---|
| (3S,2′S) I (bromide) | 60 |
| budesonide | 200 |
| lactose | 12240 |
| Total | 12500 |

2)

| | |
|---|---|
| (3S,2′S) I (bromide) | 60 |
| Etiprednol-dichloroacetate | 125 |
| lactose | 4815 |
| Total | 5000 |

3)

| | |
|---|---|
| (3S,2′S) I (bromide) | 60 |
| Mometasone-furoate X H₂O | 250 |
| lactose | 12240 |
| Total | 12500 |

4)

| | |
|---|---|
| (3S,2′S) I (bromide) | 60 |
| Ciclesonide | 250 |
| lactose | 12240 |
| Total | 12500 |

5)

| | |
|---|---|
| (3S,2′S) I (bromide) | 100 |
| budesonide | 125 |
| lactose | 4775 |
| Total | 5000 |

6)

| | |
|---|---|
| (3S,2′S) I (bromide) | 100 |
| Etiprednol-dichloroacetate | 200 |
| lactose | 4700 |
| Total | 5000 |

7)

| | |
|---|---|
| (3S,2′S) I (bromide) | 100 |
| Mometasone-furoate X H₂O | 250 |
| lactose | 4650 |
| Total | 5000 |

8)

| | |
|---|---|
| (3S,2′S) I (bromide) | 100 |
| Ciclesonide | 250 |
| lactose | 4650 |
| Total | 5000 |

B) Propellant-Containing Aerosols for Inhalation:

| | |
|---|---|
| Ingredients | % by weight |

1)

| | |
|---|---|
| (3S,2′S) I (bromide) | 0.15 |
| budesonide | 0.4 |
| soya lecithin | 0.2 |
| TG 134a:TG227 = 2:3 | ad 100 |

2)

| | |
|---|---|
| (3S,2′S) I (bromide) | 0.2 |
| Etiprednol-dichloroacetate | 0.3 |
| Isopropyl myristate | 0.1 |
| TG 227 | ad 100 |

3)

| | |
|---|---|
| (3S,2′S) I (bromide) | 0.15 |
| Mometasone-furoate X H₂O | 0.6 |
| Isopropyl myristate | 0.1 |
| TG 227 | ad 100 |

4)

| | |
|---|---|
| (3S,2′S) I (bromide) | 0.2 |
| Ciclesonide | 0.4 |
| Isopropyl myristate | 0.1 |
| TG 134a:TG227 = 2:3 | ad 100 |

Inhalable Powders (3R,2′S):

| | |
|---|---|
| Ingredients | μg per capsule |

1)

| | |
|---|---|
| (3R,2′S) I (bromide) | 60 |
| budesonide | 200 |
| lactose | 12240 |
| Total | 12500 |

2)

| | |
|---|---|
| (3R,2′S) I (bromide) | 60 |
| Etiprednol-dichloroacetate | 125 |
| lactose | 4815 |
| Total | 5000 |

3)

| | |
|---|---|
| (3R,2′S) I (bromide) | 60 |
| Mometasone-furoate X H₂O | 250 |
| lactose | 12240 |
| Total | 12500 |

4)

| | |
|---|---|
| (3R,2′S) I (bromide) | 60 |
| Ciclesonide | 250 |
| lactose | 12240 |
| Total | 12500 |

5)

| | |
|---|---|
| (3R,2′S) I (bromide) | 100 |
| budesonide | 125 |
| lactose | 4775 |
| Total | 5000 |

6)

| | |
|---|---|
| (3R,2′S) I (bromide) | 100 |
| Etiprednol-dichloroacetate | 200 |
| lactose | 4700 |
| Total | 5000 |

7)

| | |
|---|---|
| (3R,2′S) I (bromide) | 100 |
| Mometasone-furoate X H₂O | 250 |
| lactose | 4650 |
| Total | 5000 |

8)

| | |
|---|---|
| (3R,2′S) I (bromide) | 100 |
| Ciclesonide | 250 |
| lactose | 4650 |
| Total | 5000 |

B) Propellant-Containing Aerosols for Inhalation:

| | |
|---|---|
| Ingredients | % by weight |

1)

| | |
|---|---|
| (3R,2′S) I (bromide) | 0.15 |
| budesonide | 0.4 |
| soya lecithin | 0.2 |
| TG 134a:TG227 = 2:3 | ad 100 |

2)

| | |
|---|---|
| (3R,2′S) I (bromide) | 0.2 |
| Etiprednol-dichloroacetate | 0.3 |
| Isopropyl myristate | 0.1 |
| TG 227 | ad 100 |

3)

| | |
|---|---|
| (3R,2′S) I (bromide) | 0.15 |
| Mometasone-furoate X H₂O | 0.6 |
| Isopropyl myristate | 0.1 |
| TG 227 | ad 100 |

4)

| | |
|---|---|
| (3R,2′S) I (bromide) | 0.2 |
| Ciclesonide | 0.4 |
| Isopropyl myristate | 0.1 |
| TG 134a:TG227 = 2:3 | ad 100 |

Inhalable Powders (3R,2′R):

| | |
|---|---|
| Ingredients | μg per capsule |

1)

| | |
|---|---|
| (3R,2′R) I (bromide) | 60 |
| budesonide | 200 |
| lactose | 12240 |
| Total | 12500 |

2)

| | |
|---|---|
| (3R,2′R) I (bromide) | 60 |
| Etiprednol-dichloroacetate | 125 |
| lactose | 4815 |
| Total | 5000 |

3)

| | |
|---|---|
| (3R,2′R) I (bromide) | 60 |
| Mometasone-furoate X H₂O | 250 |
| lactose | 12240 |
| Total | 12500 |

4)

| | |
|---|---|
| (3R,2′R) I (bromide) | 60 |
| Ciclesonide | 250 |
| lactose | 12240 |
| Total | 12500 |

5)

| | |
|---|---|
| (3R,2′R) I (bromide) | 100 |
| budesonide | 125 |
| lactose | 4775 |
| Total | 5000 |

6)

| | |
|---|---|
| (3R,2′R) I (bromide) | 100 |
| Etiprednol-dichloroacetate | 200 |
| lactose | 4700 |
| Total | 5000 |

7)

| | |
|---|---|
| (3R,2′R) I (bromide) | 100 |
| Mometasone-furoate X H₂O | 250 |
| lactose | 4650 |
| Total | 5000 |

8)

| | |
|---|---|
| (3R,2′R) I (bromide) | 100 |
| Ciclesonide | 250 |
| lactose | 4650 |
| Total | 5000 |

B) Propellant-Containing Aerosols for Inhalation:

| | |
|---|---|
| Ingredients | % by weight |

1)

| | |
|---|---|
| (3R,2′R) I (bromide) | 0.15 |
| budesonide | 0.4 |
| soya lecithin | 0.2 |
| TG 134a:TG227 = 2:3 | ad 100 |

2)

| | |
|---|---|
| (3R,2′R) I (bromide) | 0.2 |
| Etiprednol-dichloroacetate | 0.3 |
| Isopropyl myristate | 0.1 |
| TG 227 | ad 100 |

3)

| | |
|---|---|
| (3R,2′R) I (bromide) | 0.15 |
| Mometasone-furoate X H₂O | 0.6 |
| Isopropyl myristate | 0.1 |
| TG 227 | ad 100 |

4)

| | |
|---|---|
| (3R,2′R) I (bromide) | 0.2 |
| Ciclesonide | 0.4 |
| Isopropyl myristate | 0.1 |
| TG 134a:TG227 = 2:3 | ad 100 |

What is claimed is:

1. A Pharmaceutical composition, which comprises as a first active substance one or more salts of formula 1 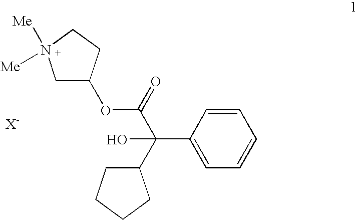
wherein
X⁻ denotes an anion with a single negative charge, preferably an anion selected from the group consisting of fluoride, chloride, bromide, iodide, sulphate, phosphate, methanesulphonate, nitrate, maleate, acetate, citrate, fumarate, tartrate, oxalate, succinate, benzoate and p-toluenesulphonate, optionally in the form of the racemates, the stereoisomers, and the hydrates thereof, and one or more steroids as a second active substance.

2. The pharmaceutical composition of claim 1 wherein the second active substance is selected from the group consisting of methyl prednisolone, prednisone, butixocort propionate, RPR-106541, flunisolide, triamcinolone, budesonide, mometasone, ciclesonide, rofleponide, ST-126, dexamethasone, 6α,9α-difluoro-17α-[(2-furanylcarbonyl)oxy]-11β-hydroxy-16α-methyl-3-oxo-androsta-1,4-diene-17β-carbothioic acid (S)-fluoromethyl ester, 6α,9α-difluoro-11β-hydroxy-16α-methyl-3-oxo-17α-propionyloxy-androsta-1,4-diene-17β-carbothioic acid (S)-(2-oxo-tetrahydro-furan-3S-yl) ester, and etiprednol, optionally in the form of the stereoisomers, mixtures of the stereoisomers or in the form of the racemates thereof, optionally in the form of the solvates or hydrates.

3. The pharmaceutical composition according to claim 1, characterised in that the first active substance and the second active substance are present together in a single formulation or in two separate formulations.

4. The pharmaceutical composition according to claim 1, characterised in that the compounds of formula 1 are present in form of the (3S,2′R)-diasteromer, in form of the (3S,2′S)-diasteromer, in form of the (3R,2′S)-diasteromer or in form of the (3R,2′R)-diasteromer.

5. The pharmaceutical composition according to claim 2, characterised in that the second active substance steroid is selected from the group consisting of flunisolide, triamcinolone, budesonide, mometasone, ciclesonide, rofleponide, ST-126, dexamethasone, 6α,9α-difluoro-17α-[(2-furanylcarbonyl)oxy]-11β-hydroxy-16α-methyl-3-oxo-androsta-1,4-diene-17β-carbothioic acid (S)-fluoromethyl ester, 6α,9α-difluoro-11β-hydroxy-16α-methyl-3-oxo-17α-propionyloxy-androsta-1,4-diene-17β-carbothioic acid (S)-(2-oxo-tetrahydro-furan-3S-yl) ester, and etiprednol.

6. The pharmaceutical composition according to claim 5, characterised in that the second active substance steroid is selected from the group consisting of budesonide, mometasone, ciclesonide, 6α,9α-difluoro-17α-[(2-furanylcarbonyl)oxy]-11β-hydroxy-16α-methyl-3-oxo-androsta-1,4-diene-17β-carbothioic acid (S)-fluoromethyl ester, and etiprednol.

7. The pharmaceutical composition according to claim 1, characterised in that the weight ratios of first active substance to second active substance are in a range from about 1:250 to 250:1.

8. The pharmaceutical composition according to claim 1, characterised in that it is in the form of a preparation suitable for inhalation.

9. The pharmaceutical composition according to claim 8, characterised in that it is a preparation selected from among the inhalable powders, propellant-containing metered-dose aerosols and propellant-free inhalable solutions.

10. The pharmaceutical composition according to claim 9, characterised in that it is an inhalable powder which comprises the first active substance and the second active substance in admixture with suitable physiologically acceptable excipients selected from among the monosaccharides, disaccharides, oligo- and polysaccharides, polyalcohols, salts, or mixtures of these excipients with one another.

11. The pharmaceutical composition according to claim 10, characterised in that the excipient has a maximum average particle size of up to 250 μm.

12. The pharmaceutical composition according to claim 9, characterised in that it is an inhalable powder which consists of only the first active substance and the second active substance as its ingredients.

13. The pharmaceutical composition according to claim 9, characterised in that it is a propellant-containing inhalable aerosol which contains the first active substance and the second active substance in dissolved or dispersed form.

14. The pharmaceutical composition according to claim 13, characterised in that it comprises as propellant gas hydrocarbons, such as n-propane, n-butane or isobutene, or halohydrocarbons, such as chlorinated and/or fluorinated derivatives of methane, ethane, propane, butane, cyclopropane or cyclobutane.

15. The pharmaceutical composition according to claim 14, characterised in that the propellant gas is TG11, TG12, TG134a, TG227 or mixtures thereof.

16. The pharmaceutical composition according to claim 9, characterised in that it is a propellant-free inhalable solution which comprises water, ethanol or a mixture of water and ethanol as solvent.

17. The pharmaceutical composition according to claim 16, characterised in that it comprises as co-solvents ingredients which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters.
